(19) United States
(12) Patent Application Publication
(10) Pub. No.: US 2011/0029001 A1
(43) Pub. Date: Feb. 3, 2011

(54) SYSTEM AND METHOD FOR ANNULUS TREATMENT

(75) Inventors: Hai H. Trieu, Cordova, TN (US); Jeff Justis, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/511,659

(22) Filed: Jul. 29, 2009

(51) Int. Cl. A61B 17/08

(52) U.S. Cl. 606/151; 606/213

(57) ABSTRACT

A system for treating an intervertebral disc includes a plurality of self-closing clips including a first clip and a second clip being deployable adjacent to an opening of the intervertebral disc. The first clip and the second clip are fixed with tissue about the opening and configured to prevent undesired cutting or tearing of the tissue. The clips are disposed in a configuration to facilitate closure of the opening. A suture member is connected to the first clip and the second clip in a configuration such that the suture member is manipulable to draw the opening to a closed position. A locking part is connected to the suture member and is configured to maintain the opening in the closed position. Methods of use are also disclosed.

### TECHNICAL FIELD

The present disclosure generally relates to medical devices, systems and methods for the treatment of intervertebral discs, and more particularly to methods and systems that employ clips, which are configured to prevent undesired cutting or tearing of tissue, for the closure, sealing, repair and/or reconstruction of an intervertebral disc annulus.

### BACKGROUND

Intervertebral discs lie between adjacent vertebrae of a spine. An intervertebral disc includes fibrosus bands, which provide cushion to facilitate motion of the vertebrae and spacing of the vertebrae from nerves and vessels. The fibrosus bands include an outer annulus fibrosus, which is the peripheral portion of an intervertebral disc and defines an inner nucleus pulposus. The nucleus pulposus includes loose fibers suspended in a gel substance having a jelly like consistency that absorbs impacts to the body while keeping the vertebrae separated.

The annulus fibrosus is susceptible to tearing due to injury and/or the aging process. Defects such as tears, fissures or rupture in the annulus fibrosus may cause disc herniation whereby the nucleus pulposus, which can include inflammatory cytokines, leaks or migrates from the intervertebral disc into the body. Typically, the herniated disc compresses the nerves and/or other vessels resulting in chronic and debilitating pain. Further, the leakage of nucleus pulposus may contact the nerves, which can cause significant pain.

Known surgical treatments for such defects of the annulus can include discectomy, laminectomy, fusion, implantable prosthetics and disc herniation repair. However, these surgical treatments may result in the creation of defects such as incisions or openings in the annulus, which may also require closure, sealing, repair and/or reconstruction.

In some cases, the annulus defects may be left unrepaired, which can lead to disc reherniation or implant expulsion from the disc space. In other cases, annulus repair is sought via sutures that attempt to close the defect by pulling the surrounding tissue together. However, there can be several drawbacks associated with this attempt to repair the annulus. For example, the annulus defect may be an opening that is difficult to close with conventional suturing techniques due to the size and/or configuration of the opening. It can also be difficult to actively engage the sutures in the surrounding annulus tissues, and/or the sutures could cut or tear through the annulus tissues during and after treatment.

Therefore, it would be desirable to provide methods and systems that employ self-closing clips, which are configured to prevent undesired cutting or tearing of tissue, for the closure, sealing, repair and/or reconstruction of an intervertebral disc annulus. It would be desirable if the clips are deployed adjacent to a treatment site in a predetermined configuration to facilitate closure of a defect of the annulus. It is contemplated that the present systems and methods may also employ an occluding member to treat an annulus defect.

### SUMMARY OF THE INVENTION

Accordingly, the present disclosure provides methods and systems that employ self-closing clips, which are configured to prevent undesired cutting or tearing of tissue, for the closure, sealing, repair and/or reconstruction of an intervertebral disc annulus. Desirably, the clips are deployed adjacent to a treatment site in a predetermined configuration to facilitate closure of a defect of the annulus. The present systems and methods may also employ an occluding member to treat an annulus defect.

In one particular embodiment, in accordance with the principles of the present disclosure, a system for treating an intervertebral disc is provided. The system includes a plurality of self-closing clips including a first clip and a second clip being deployable adjacent to an opening of the intervertebral disc. The first clip and the second clip are fixed with tissue about the opening and configured to prevent undesired cutting or tearing of the tissue. The clips are disposed in a configuration to facilitate closure of the opening. A suture member is connected to the first clip and the second clip in a configuration such that the suture member is manipulable to draw the opening to a closed position. A locking part is connected to the suture member and is configured to maintain the opening in the closed position.

Alternatively, the plurality of self-closing clips include a third clip and a fourth clip that are deployable adjacent to the opening. The third clip and the fourth clip are fixed with the tissue about the opening and configured to prevent undesired cutting or tearing of the tissue. The suture member is connected to the third clip and the fourth clip in a configuration such that the suture member is manipulable to draw the opening to the closed position.

The plurality of self-closing clips may also include a fifth clip and a sixth clip that are deployable adjacent to the opening. The fifth clip and the sixth clip are fixed with the tissue about the opening and configured to prevent undesired cutting or tearing of the tissue. The suture member is connected to the fifth clip and the sixth clip in a configuration such that the suture member is manipulable to draw the opening to the closed position.

In an alternate embodiment, the system includes a first self-closing clip that is deployable adjacent to an opening of the intervertebral disc. The clip is fixed with tissue about the opening and configured to prevent undesired cutting or tearing of the tissue. An occlusion member is disposable within the opening and fixed with the clip in a configuration such that the occlusion member is attached with the tissue.

The occlusion member may be fixed with the clip via a first suture member. A second self-closing clip may be deployable to the opening. The second clip is fixed with tissue about the opening and configured to prevent undesired cutting or tearing of the tissue. The occlusion member is fixed with the second clip via a second suture member.

In another embodiment, a method for treating an intervertebral disc is provided. The method includes the steps of: providing a plurality of self-closing clips including a first clip and a second clip, and a suture member being connected to the first clip and the second clip; deploying the first clip and the second clip in fixed engagement with tissue about the opening in a configuration to facilitate closure of the opening, the clips being fixed with the tissue about the opening and configured to prevent undesired cutting or tearing of the tissue; manipulating the suture member to draw the opening to a closed position; and connecting a locking part to the suture member adjacent to the clips to maintain the opening in the closed position.

Alternatively, the method may include providing and deploying the plurality of self-closing clips with a third clip and a fourth clip, and the suture member is connected to the third clip and the fourth clip. The method may also include providing and deploying the plurality of self-closing clips with a fifth clip and a sixth clip, and the suture member is connected to the fifth clip and the sixth clip.

In another embodiment, the method for treating an intervertebral disc may include the steps of: providing an occlusion member; delivering the occlusion member adjacent to an opening of the intervertebral disc for disposal therein; providing a first self-closing clip; delivering the clip adjacent to the opening; and deploying the first clip in fixed engagement with tissue about the opening and in fixed engagement with the occlusion member such that the occlusion member is attached with the tissue, the first clip being fixed with tissue about the opening and configured to prevent undesired cutting or tearing of the tissue.

Alternatively, the occlusion member is fixed with the first clip via a first suture member, and the method further includes the step of connecting a locking part to the first suture member adjacent to the first clip to maintain the occlusion member within the opening.

The method may also include the steps of: providing a second self-closing clip; delivering the second clip adjacent to the opening; and deploying the second clip in fixed engagement with the tissue about the opening and in fixed engagement with the occlusion member such that the occlusion member is attached with the tissue, the second clip being fixed with tissue about the opening and configured to prevent undesired cutting or tearing of the tissue.

In one embodiment, the present disclosure relates to a method and apparatus for closing an annular defect. The apparatus includes at least a suture member and two U-clips. The apparatus may further include additional U-clips. An example of the suture member includes a wound-closing suture, a thread, a wire, a tether and/or a cable in selected size or shape. An example of the U-clip includes a shape-memory or deployable nitinol loop, ring and/or staple.

In another embodiment, the present disclosure relates to a method including the steps of: delivering and deploying the U-clips (with attached suture member) such that they are located and/or secured around the annular defect; pulling the suture member to close up the annular defect without pulling the U-clips off the annular tissue; and tying a knot and/or securing the suture with a locking button to hold a closing position. The U-clips can be delivered and deployed by a surgical connection apparatus. See, for example, those surgical connection apparatus and methods disclosed in U.S. Patent Application Publication No. 2005/0070924, the contents of which are hereby incorporated by reference herein, in its entirety. The present disclosure provides a U-clip that does not bridge across an annular defect, but rather the U-clip described herein is used to anchor the suture member.

In another embodiment, the systems and methods of the present disclosure may include an additional element, such as, for example, an occluding member. The system of the present disclosure can include at least one occlusion member and a U-clip. The system may further comprise a suture connecting the occlusion member and the U-clip. An example of the occlusion member includes a plug, sheet, sponge and/or mesh in selected size or shape. An example of the U-clip includes a shape-memory or deployable nitinol loop, ring and/or staple. In another embodiment, the present disclosure relates to a method including the steps of: delivering the occlusion member through the annular defect; deploying the U-clip such that it connects and/or secures the occlusion member with the adjacent annular tissue; and alternatively, using suture to connect or secure the occlusion member to the U-clip. The system and method may employ a suture, a knot or locking button for securing the apparatus together. It is preferred that more than one U-clip is used to secure the occlusion member to the adjacent annular tissues. The U-clips can be delivered and deployed by a surgical connection apparatus. The present disclosure provides a U-clip that does not bridge across the annular defect, but rather is used to anchor the occlusion member.

### BRIEF DESCRIPTION OF THE DRAWINGS

The present disclosure will become more readily apparent from the specific description accompanied by the following drawings, in which:

FIG. 1 is a perspective view of a clip employed with one particular embodiment of a system and method for treating an intervertebral disc in accordance with the principles of the present disclosure;

FIG. 2 is a perspective view of a spine section including an intervertebral disc annulus having an opening;

FIG. 3 is a plan view of the opening of the annulus shown in FIG. 2 being treated with the presently disclosed system and method;

FIG. 4 is a top view of the opening of the annulus shown in FIG. 3 being treated with the presently disclosed system and method, with the opening being drawn to the closed position;

FIG. 5 is a plan view of the annulus shown in FIG. 3 being treated with the presently disclosed system and method, with the opening being drawn to the closed position;

FIG. 6 is a side view of the opening of the annulus shown in FIG. 2 being treated with an alternate embodiment of the presently disclosed system and method; and

FIG. 7 is a plan view of the annulus shown in FIG. 6 being treated with the presently disclosed system and method, with an occluding member closing the opening.

Like reference numerals indicate similar parts throughout the figures.

### DETAILED DESCRIPTION OF THE INVENTION

The exemplary embodiments of the subject invention are discussed in terms of medical devices, systems and methods for the treatment of intervertebral discs and more particularly, in terms of methods and systems that employ clips, which are configured to prevent undesired cutting or tearing of tissue, for the closure, sealing, repair and/or reconstruction of an intervertebral disc annulus and accompanying delivery devices and tools, and their methods of use. Treatment can include repair of an aperture in the intervertebral disc wall, or a weakened or thin portion thereof. An aperture of the intervertebral disc annulus can include an opening in the annulus that is a result of a surgical incision or dissection into the intervertebral disc annulus, or the consequence of a naturally occurring tear or defect. The present system and method may also be employed to treat other portions of the spine such as vertebrae and surrounding tissues.

The system of the subject invention may include an annular repair device and/or minimally invasive instruments to facilitate access to a treatment site. It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft and implantable prosthetics.

It is contemplated that the present disclosure may be employed with other osteal and bone related applications, including those associated with diagnostics and therapeutics. It is further contemplated that the apparatus and methods of the subject invention may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

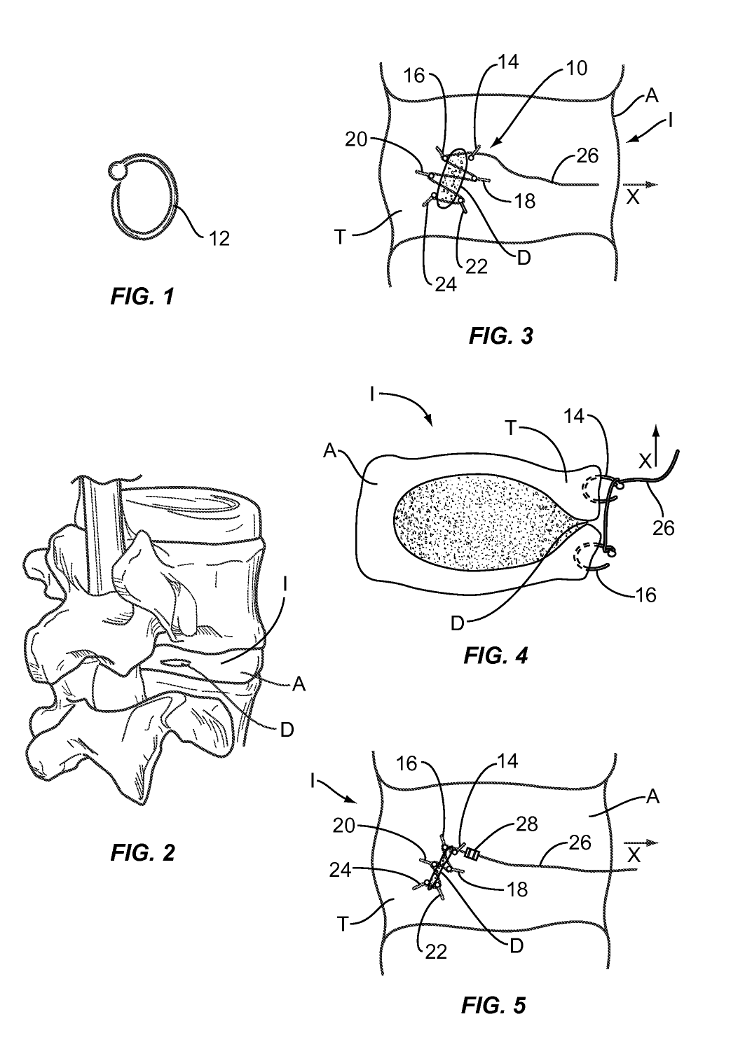

The following discussion includes a description of system for treating an intervertebral disc, related components and exemplary methods of employing the system for treating an intervertebral disc in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a system 10 for treating an intervertebral disc I in accordance with the principles of the present disclosure.

The components of system 10 for treating an intervertebral disc can be generally fabricated from materials suitable for medical applications, including metals, polymers, ceramics, biocompatible materials and/or their composites, depending on the particular application and/or preference of a medical practitioner. One skilled in the art, however, will realize that such materials and fabrication methods suitable for the components of the present system, in accordance with the present disclosure, would be appropriate.

System 10 is configured and assembled for treating an intervertebral disc annulus A. System 10 includes a plurality of self-closing clips 12 including a first clip 14, a second clip 16, a third clip 18, a fourth clip 20, a fifth clip 22 and a sixth clip 24. Clips 14-24 are deployable adjacent to an opening, such as, for example, defect D of annulus A.

Clips 12 are movable and self closing between an open configuration and a closed configuration for connection to tissue. See, for example, those clips and methods disclosed in U.S. Pat. Nos. 6,514,265, 6,641,593, 6,607,541 and U.S. Patent Application Publication No. 2007/0027461, the contents of each of these references being hereby incorporated by reference herein, in their entireties. Clips 12 are employed thereafter to close a defect or other opening in the tissue by drawing the clips together. Clips 12 are movable to a linear configuration for loading with a surgical connection apparatus. Clips 12 have a generally arcuate shaped configuration in its closed configuration to facilitate retention of the clip with the tissue. It is contemplated that clips 12 may be alternatively configured such as U-shaped, C-shaped, V-shaped or J-shaped in the closed configuration and/or the open configuration.

Clips 14-24 are fixable with tissue T about defect D and configured to prevent undesired cutting or tearing of tissue T during treatment. Clips 14-24 are configured to prevent undesired cutting or tearing of tissue T in that each of clips 14-24 have a cross-section geometry that avoids damaging tissue, such as, for example, circular, elliptical, polygonal with planar faces and a cross-sectional diameter or thickness in the range of 50-1500 microns and preferably in the range of 100-500 microns. It is contemplated that the present system and method can employ clips having a wire/coil configuration having a cross-sectional diameter or thickness of the wire in the range of 25-500 microns and preferably in the range of 50-250 microns, and a cross-sectional diameter or thickness of the coil in the range of 50-1000 microns and preferably in the range of 100-500 microns.

It is contemplated that system 10 may employ one or a plurality of fasteners and/or clips to treat defect D. It is further contemplated that defect D may include an aperture of annulus A due to a weakened or thinned portion thereof, an opening from a surgical incision or dissection into annulus A, and/or the consequence of a naturally occurring or injury related tear or defect.

Plurality of self-closing clips 12 can be fabricated from materials such as a rigid, semi-rigid, elastic, flexible and/or a shape memory alloy including a nickel titanium (nitinol) based alloy. The nitinol may include additional elements, which affect the yield strength of the material or the temperature at which particular pseudoelastic or shape transformation characteristics occur. It is contemplated that the shape memory alloy may also be heat activated, or a combination of heat activation and pseudoelastic properties may be used. Plurality of self-closing clips 12 can also be plastically deformable and/or fabricated from a plastically deformable material.

Clips 14-24 are deployed for arrangement in a configuration to facilitate closure of defect D. Clips 14-24 are deployed about defect D in a predetermined configuration such that clips 14-24 are disposed in a circular orientation or layout about defect D to facilitate closure of defect D. It is envisioned that clips 12 may be deployed in an alternate plan layout or orientation such as, for example, rectangular, elliptical, linear, undulating and offset.

Clips 14-24 may be configured as a deployable nitinol loop, ring and/or staple. The clip may have different structural sections that may include alternative material composites to achieve various desired characteristics such as strength, rigidity, elasticity, compliance, biomechanical performance, durability and radiolucency or imaging preference. In an alternate embodiment, clips 14-24 are fabricated from a material having a minimum shear and/or tensile strength such that the clips are deployable into bone. In another alternate embodiment, clips 14-24 are fabricated from a shape memory alloy having a minimum shear and/or tensile strength such that the clips are deployable into bone. System 10 provides clips having greater strength such that the stronger clips may be deployed into bone directly, and the clips of lesser strength may be deployed into other tissues.

A suture member 26 is connected to clips 14-24 in a configuration such that suture member 26 is manipulable, in the direction shown by arrow X shown in FIGS. 3-5, to draw defect D to a closed position, as shown in FIGS. 4 and 5. It is envisioned that one or a plurality of suture members may be employed with plurality of clips 12. It is further envisioned that suture member 26 may be employed with only first clip 14 and second clip 16. Suture member 26 may be fabricated from materials such as conventional filament material, a metal alloy such as nitinol, polymeric material, or any other suitable material. The material may be non-stretchable or stretchable, solid or hollow, and have various cross-sectional diameters. The diameter and length of the suture can vary depending on the specific application. The suture member may be attached to a clip of the present invention by crimping or swaging the clip onto the suture member, or any other suitable attachment method. It is contemplated that suture member 26 may include a wound-closing suture, a thread, a wire, a tether and/or a cable in selected size or shape according to the particular application.

A locking part 28 is connected to suture member 26 and is configured to maintain defect D in the closed position. Locking part 28 may include a knot, lock, fastener, clip, retainer, and/or other securing device that holds the drawn together position of clips 14-24 such that defect D is forced into a closed position for treatment thereof.

In use, system 10 is employed with a method for treating intervertebral disc I. System 10 is introduced into the soft tissue of a patient to the targeted intervertebral disc annulus A to treat defect D.

To treat the affected annulus A, a medical practitioner obtains access to a surgical site including the intervertebral disc in any appropriate manner, such as through incision and retraction of tissues. It is envisioned that system 10 may be used in any existing surgical method or technique including open surgery, mini-open surgery, minimally invasive surgery and percutaneous surgical implantation, whereby the intervertebral disc is accessed through a micro-incision, or sleeve that provides a protected passageway to the area. Once access to the surgical site is obtained, the particular surgical procedure employing system 10 is performed for treating the spinal disorder.

System 10, including self-closing clips 12, is delivered to the surgical site for treating defect D. Clips 14-24 are deployed by a surgical connection apparatus (see, for example, U.S. Patent Application Publication No. 2005/0070924) about defect D in a predetermined configuration such that clips 14-24 are disposed in a circular orientation about defect D. Suture member 26 is connected to clips 14-24.

First clip 14 is deployed to a first predetermined site location adjacent defect D by the surgical connection apparatus in fixed engagement with tissue T about defect D in a configuration to facilitate closure of defect D. Second clip 16 is deployed to a second predetermined site location adjacent defect D by the surgical connection apparatus in fixed engagement with tissue T about defect D in a configuration to facilitate closure of defect D. Clips 18-24 are similarly deployed to third through sixth predetermined site locations, respectively, adjacent defect D by the surgical connection apparatus in fixed engagement with tissue T about defect D in a configuration to facilitate closure of defect D. The predetermined site locations are formulated based on a suitable distance from defect D such that deployment of clips 14-24 does not damage tissue T, and such that clips 14-24 are disposed in a circular orientation about defect D to facilitate closure of defect D according to the requirements of a particular application.

The medical practitioner manipulates suture member 26, which is connected to clips 14-24, to draw defect D to a closed position, as shown in FIGS. 4-5. Upon fixation and during manipulation of suture member 26, clips 14-24 are configured to prevent undesired cutting or tearing of tissue T, as discussed.

Locking part 28 is connected to suture member 26 adjacent to clip 14, and is employed to maintain defect D in the closed position, as discussed above.

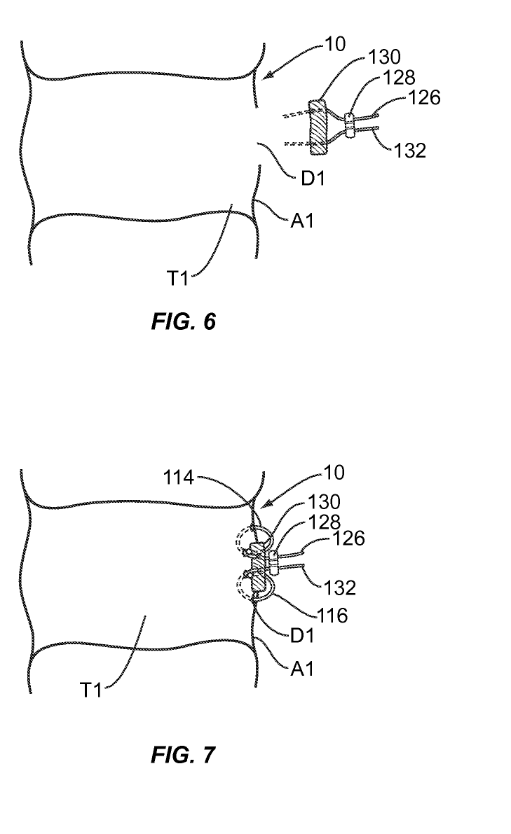

Referring to FIGS. 6 and 7, in an alternate embodiment, system 10 includes a first self-closing, U-shaped clip 114, similar to clips 12 discussed above with regard to FIGS. 1-5, which is deployable adjacent to a defect D1 of an annulus A1. Clip 114 is fixed with tissue T1 about defect D1 and configured to prevent undesired cutting or tearing of tissue T1. It is contemplated that system 10 may employ one or a plurality of clips 114 to treat defect D1.

An occlusion member 130 is disposable within defect D1 and fixed with clip 114 in a configuration such that occlusion member 130 is attached with tissue T1. Occlusion member 130 may be fabricated from various materials, which may be in the form of a plug, sheet, sponge and/or mesh in a selected size or shape according to the requirements of a particular application.

Alternatively, a locking part 128 is connected to a suture member 126, similar to those discussed with regard to FIGS. 1-5, which is connected to clip 114. Locking part 128 and suture member 126 are configured to secure occluding member 130 to clip 114 and maintain occluding member 130 within defect D.

Alternatively, system 10 may employ clip 114 and a second self-closing, U-shaped clip 116, similar to clip 114, which is deployable to defect D1. Clip 116 is fixed with tissue T1 about defect D1 and configured to prevent undesired cutting or tearing of tissue T1. Locking part 128 is connected to a suture member 132, which is connected to clip 116. Locking part 128 and suture member 132 are configured to secure occluding member 130 to clip 116 and maintain occluding member 130 within defect D.

System 10 is employed with a method for treating annulus A1, similar to the method described above, and includes occlusion member 130, which is delivered to a surgical site for treating defect D1. Occlusion member 130 is delivered to defect D1 and disposed therewithin. Self-closing, U-shaped clips 114, 116 are delivered to the surgical site for treating defect D1. Clips 114, 116 are deployed by a surgical connection apparatus to tissue T1 adjacent defect D1. Suture member 126 is connected to clip 114 and suture member 132 is connected to clip 116.

First clip 114 is deployed to a first predetermined site location adjacent defect D1 by the surgical connection apparatus in fixed engagement with tissue T1 about defect D1 and in fixed engagement with occlusion member 130 such that occlusion member 130 is attached with tissue T1. Second clip 116 is deployed to a second predetermined site location adjacent defect D1 by the surgical connection apparatus in fixed engagement with tissue T1 about defect D1 and in fixed engagement with occlusion member 130 such that occlusion member 130 is attached with tissue T1. The predetermined site locations are formulated based on a suitable distance from defect D1 such that deployment of clips 114, 116 does not damage tissue T, and occlusion member 130 closes defect D1 according to the requirements of a particular application.

Locking part 128 is connected to suture members 126, 132 adjacent to clips 114, 116, respectively, to secure occlusion member 130 within defect D1 such that defect D1 is maintained in the closed position.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A system for treating an intervertebral disc, the system comprising:
a plurality of self-closing clips including a first clip and a second clip being deployable adjacent to an opening of the intervertebral disc, the first clip and the second clip being fixed with tissue about the opening and configured to prevent undesired cutting or tearing of the tissue, the clips being disposed in a configuration to facilitate closure of the opening;
a suture member being connected to the first clip and the second clip in a configuration such that the suture member is manipulable to draw the opening to a closed position; and
a locking part connected to the suture member and being configured to maintain the opening in the closed position.

2. A system for treating an intervertebral disc according to claim 1, wherein the plurality of self-closing clips include a third clip and a fourth clip being deployable adjacent to the opening, the third clip and the fourth clip being fixed with the tissue about the opening and configured to prevent undesired cutting or tearing of the tissue, and the suture member being connected to the third clip and the fourth clip in a configuration such that the suture member is manipulable to draw the opening to the closed position.

3. A system for treating an intervertebral disc according to claim 1, wherein the plurality of self-closing clips include a fifth clip and a sixth clip being deployable adjacent to the opening, the fifth clip and the sixth clip being fixed with the tissue about the opening and configured to prevent undesired cutting or tearing of the tissue, and the suture member being connected to the fifth clip and the sixth clip in a configuration such that the suture member is manipulable to draw the opening to the closed position.

4. A system for treating an intervertebral disc according to claim 1, wherein the clips are configured to prevent undesired cutting or tearing of the tissue such that the clips have a cross-sectional thickness in the range of 50-1500 microns.

5. A system for treating an intervertebral disc according to claim 1, wherein the clips are configured to prevent undesired cutting or tearing of the tissue such that the clips have a cross-sectional thickness in the range of 100-500 microns.

6. A system for treating an intervertebral disc according to claim 1, wherein the clips include a wire having a coil disposed thereabout, the wire having a cross-sectional thickness in the range of 25-500 microns.

7. A system for treating an intervertebral disc according to claim 1, wherein the clips include a wire having a coil disposed thereabout, the wire having a cross-sectional thickness in the range of 50-250 microns.

8. A system for treating an intervertebral disc according to claim 1, wherein the clips include a wire having a coil disposed thereabout, the clips being configured to prevent undesired cutting or tearing of the tissue such that the clips have a cross-sectional thickness in the range of 50-1000 microns.

9. A system for treating an intervertebral disc according to claim 1, wherein the clips include a wire having a coil disposed thereabout, the clips being configured to prevent undesired cutting or tearing of the tissue such that the clips have a cross-sectional thickness in the range of 100-500 microns.

10. A system for treating an intervertebral disc according to claim 1, wherein the clips are deployed about the opening in a predetermined configuration such that the clips are deployed in a plan layout having a circular configuration to facilitate closure of the opening.

11. A system for treating an intervertebral disc according to claim 1, wherein the clips are deployed about the opening in a predetermined configuration such that the clips are deployed in a plan layout having an elliptical configuration to facilitate closure of the opening.

12. A system for treating an intervertebral disc according to claim 1, wherein the self-closing clips are fabricated from a material having a minimum strength such that the clips are deployable into bone.

13. A system for treating an intervertebral disc according to claim 1, wherein the self-closing clips are fabricated from a shape memory alloy.

14. A system for treating an intervertebral disc according to claim 13, wherein the shape memory alloy has a minimum strength such that the clips are deployable into bone.

15. A system for treating an intervertebral disc according to claim 1, wherein at least one clip is configured for deployment into bone and at least one clip is configured for deployment into soft tissue.

16. A system for treating an intervertebral disc according to claim 1, wherein each of the clips have a U-shaped configuration.

17. A method for treating an intervertebral disc, the method comprising the steps of:
deploying a plurality of self-closing clips including a first clip and a second clip adjacent to an opening of the intervertebral disc and with tissue about the opening in a configuration to facilitate closure of the opening, the clips being fixed with the tissue about the opening and configured to prevent undesired cutting or tearing of the tissue;
manipulating a suture member connected to the first clip and the second clip to draw the opening to a closed position; and
connecting a locking part to the suture member adjacent to the clips to maintain the opening in the closed position.

18. A method for treating an intervertebral disc according to claim 17, wherein the plurality of self-closing clips include a third clip and a fourth clip connected to the suture member, and further comprising the step of:
deploying the third clip and the fourth clip in fixed engagement with the tissue about the opening in a configuration to facilitate closure of the opening, the third clip and the fourth clip being fixed with the tissue about the opening and configured to prevent undesired cutting or tearing of the tissue.

19. A method for treating an intervertebral disc according to claim 17, wherein the plurality of self-closing clips include a fifth clip and a sixth clip connected to the suture member, and further comprising the steps of:
deploying the fifth clip and the sixth clip in fixed engagement with the tissue about the opening in a configuration to facilitate closure of the opening;
the third clip and the fourth clip being fixed with the tissue about the opening and configured to prevent undesired cutting or tearing of the tissue.

20. A system for treating an intervertebral disc, the system comprising:
a plurality of self-closing clips fabricated from a shape memory alloy, the clips including a first clip, a second clip, a third clip, a fourth clip, a fifth clip and a sixth clip, the clips being deployable adjacent to an opening of the intervertebral disc, the clips being fixed with tissue about the opening and configured to prevent undesired cutting or tearing of the tissue, the clips being disposed in a configuration to facilitate closure of the opening;
a suture member being connected to the clips in a configuration such that the suture member is manipulable to draw the opening to a closed position; and
a locking button connected to the suture member and being configured to maintain the opening in the closed position.
